(19) United States
(12) Patent Application Publication
(10) Pub. No.: US 2007/0255289 A1
(43) Pub. Date: Nov. 1, 2007

(54) Endoscopic instrument with rotational and axial motion control

(75) Inventor: Naomi L. Nakao, New York, NY (US)

Correspondence Address: COLEMAN SUDOL SAPONE, P.C., 714 COLORADO AVENUE, BRIDGE PORT, CT, 06605-1601, US

(73) Assignee: GRANIT MEDICAL INNOVATION, INC., New York, NY (US)

(21) Appl. No.: 11/433,932

(22) Filed: May 15, 2006

Related U.S. Application Data
(60) Provisional application No. 60/795,425, filed on Apr. 27, 2006.

(51) Int. Cl. A61B 17/00; A61B 17/26

(52) U.S. Cl. 606/113; 606/205

(57) ABSTRACT

An elongate medical instrument has an operative tip that is translatable and rotatable. A Translation and rotation are effectuated by the same elongate shifter member. The shifter member rotates together with a tubular catheter member about a longitudinal axis of the instrument. The simultaneous rotation enables the user to rotate a rotation control member on a handle assembly so that the operative tip rotates through the same angle as the control member. Where an electrical current is conducted to the operative tip for cauterization purposes, a rotary joint is provided with conductive capability.

### BACKGROUND OF THE INVENTION

This invention relates to medical instrumentation useful in endoscopic surgery, particularly flexible endoscopic surgery. This invention relates more specifically to an endoscopic instrument wherein orientation of an operative tip is important in carrying out a surgical procedure.

Much effort has been directed to developing medical instruments useful in endoscopic surgical procedures, having rotational and translational positioning capability. In the known instruments, a catheter or sheath is stationary relative to a handle assembly while a wire extending longitudinally through the sheath is provided with a rotational capability to facilitate orientation of an operative tip at the distal end of the wire with respect to a target tissue mass. Such an instrument is disclosed in U.S. Pat. No. 6,352,539 to Avellanet entitled “Surgical Instrument with Rotatable Shaft” (2002).

U.S. Pat. No. 5,084,054 to Bencini et al. entitled “Surgical Gripping Instrument” (1992) discloses an instrument where there is only relative axial motion between a sheath or catheter and a wire inserted therethrough, while U.S. Pat. No. 6,235,026 to Smith entitled “Polypectomy Snare Instrument” (2001) discloses a surgical instrument where a shaft or wire is axially and rotationally movable. Pursuant to the disclosure of the latter patent, the sheath does not rotate with the shaft or wire. Instead, the sheath remains stationary while the shaft or wire is rotated.

U.S. Pat. No. 5,788,710 to Bates et al. entitled “Calculus Removal” (1998) teaches a wire or rod inserted through a sheath of catheter and fixed to an actuator knob protruding from the proximal end of the instrument handle assembly. The knob is alternately pushed and pulled to effectuate an axial motion of the wire or rod relative to a sheath or catheter and is turned to effectuate a rotation of the wire or rod relative to the sheath of catheter.

U.S. Pat. No. 5,066,295 to Kozak et al. entitled “Rotatable Surgical Snare” (1991) is directed to a quick-release connector that couples a sheath and wire assembly to a handle assembly. The wire rotates relative to the sheath.

U.S. Pat. No. 3,955,578 to Chamness et al. entitled “Rotatable Surgical Snare” (1976) represents an early example of snare instrument with rotational capability. The snare is rotatably and axially moved by a wire that rotates and translates relative to a sheath or catheter through which the wire extends.

U.S. Pat. No. 6,602,262 to Griego et al. entitled “Medical Device Having Linear to Rotation Control” (2003) discloses an instrument where a rotational movement of a snare is accomplished by converting an axial or longitudinal motion of a shifter element into a rotary motion. U.S. Patent Publication No. 2003/0050638 by Yachia et al. entitled “Surgical Instrument and Method of Using Same” (2003) describes a surgical instrument having a rotating operative tip where the rotation is induced by a shaft traversing a positioning tube that essentially remains stationary relative to the patient during the rotary ablation.

U.S. Patent Publication No. 2003/0009276 by Bilitz entitled “Medical Retrieval Device with Independent Rotational Means” (2003) discloses an instrument with an operative tip in the form of a basket and with a mechanism for precisely rotating the basket that is independent of the extension and retraction actuation mechanism used to collapse and expand the basket. This permits rotation of the basket without requiring the user to rotate the entire handle assembly and also permits rotation of the basket without requiring the sheath to rotate within the working channel of the endoscope.

U.S. Pat. No. 5,984,920 to Steinbach entitled “Rotatable Sphincterotome/Pailltome and Method of Use” (1999) discloses a variation of the common operative configuration. In this variation, the wire is connected to the distal end of the catheter and bends or curves the distal end portion thereof when the wire is pulled in the proximal direction. Rotating the wire turns the distal end of the catheter slightly to one side of the other.

U.S. Pat. No. 6,827,718 to Hutchins et al entitled “Method and Apparatus for Positioning and Maintaining the Position of Endoscopic Instruments” (2004) similarly discloses a rotatable coupling attaching a handle to a catheter allowing the handle to rotate relative to the proximal end of the catheter while engaging and rotating a proximal end of a cutting wire whereby a distal portion of the catheter rotates as a result of the rotation of the handle. A cutting wire extending through the catheter is attached at a distal end to the distal end of the catheter so that turning of the wire relative to the catheter at the proximal end induces a turning of the catheter at the distal end.

U.S. Pat. No. 5,094,247 to Hernandez et al. entitled “Biopsy Forceps with Handle Having a Flexible Coupling” (1992) relates to a medical instrument where a handle is pivotable with respect to a secondary handle called a hub. The pivoting enables the operator to orient the handle at an angle with respect to that portion of the instrument which is inserted into a patient. The coupling includes a universal joint having a pair of orthogonal pivot axes.

### OBJECTS OF THE INVENTION

It is an object of the present invention to provide a medical instrument wherein an operative tip at the distal end of the instrument can rotate relative to a handle assembly at a proximal end of the instrument.

Another object of the present invention is to provide such a medical that may be inserted through a working channel of a flexible endoscope so that the operative tip is axially and rotationally shiftable relative to the handle assembly.

A further object of the present invention is to provide such an instrument that is easy to use.

A more particular object of the present invention, and one that differentiates it from the inventions mentioned above, is to provide such an instrument that is simpler and less expensively manufactured, despite the complex requirements of rotational, and axial movement of the end effector relative to the endoscope.

A more particular object of the present invention is to provide such an instrument where the operative tip is a cauterization loop or snare, or a hot biopsy forceps so that electrical current may be conducted along a shifter wire to the loop or snare from the handle assembly.

Yet another more particular object of the invention is to provide such an instrument where the operative tip performs an operation other than a cautery operation, such as in a grasper, capture net, or stapler device, etc.

These and other objects of the invention will be apparent from the drawings and descriptions herein. Although every object of the invention is believed to be achieved by at least one embodiment of the invention, there is not necessarily any single embodiment that achieves all of the objects of the invention.

### SUMMARY OF THE INVENTION

The present invention is directed to a medical instrument wherein a wire passing longitudinally through a sheath or catheter is axially shiftable relative to the sheath or catheter but where the sheath or catheter rotates with the wire to effectuate a rotation of the operative tip of the instrument. Axially shifting of the wire is effectuated via a ball-and socket universal joint that permits rotation of the sheath and wire relative to a handle assembly, while accommodating axial motion of the wire relative to the sheath or catheter, as well as relative to the handle assembly.

A medical instrument comprises, in accordance with the present invention, a handle assembly including a main body, an axial position control member translatable relative to the main body, and a rotation control member rotatable relative to the main body. An elongate tubular member is connected at a proximal end to the handle assembly and an elongate shifter member (a rod, cable or tubular member) longitudinally traverses the tubular member. An operative tip is connected to the shifter member at a distal end thereof. The tubular member is operatively connected to the rotation control member for rotating about a longitudinal axis in response to manipulation of the rotation control member. The shifter member is also operatively connected to the rotation control member for rotating together with the tubular member about the longitudinal axis in response to manipulation of the rotation control member. The shifter member is operatively connected to the axial position control member for shifting axially relative to the tubular member in response to manipulation of the axial position control member. Thus, the shifter member (e.g., a wire or flexible rod) is rotationally entrained to the tubular member (e.g., sheath or catheter), but is axially movable relative to the tubular member. The shifter member is rotationally fixed to the tubular member, at the proximal ends thereof, but axially or longitudinally movable relative to the tubular member.

Preferably, the shifter cable is made of a 1:1 torquable stainless steel (i.e., a rotation induced at the proximal end of the cable resulting in a rotational movement through substantially the same angle at the distal end of the cable). If the shifter member is a flexible solid rod made of NiTi (Nitinol), than the shifter member may be coated with gold or another metal that conducts well, in order to provide for the transmission of a cauterizing current. Alternatively, if the shifter member is a braided cable, then it may be made entirely of stainless steel strands constructed in a particular manner (produced on the market, and sold freely), or be made of strands of Nitinol, or another metal with 1:1 rotatable properties, and have a stainless steel wire woven into it for carrying an electrical current. If the shifter member is a tube, a wire of stainless steel or other conductive material may pass through the tube for carrying the cauterizing current. In any case, electrically conductive coating, strand, thread or wire extends along the shifter member, or makes up the entire shifter member from a proximal end to a distal end thereof. The coating, strand, thread or wire is connected to the operative tip for conveying electrical current thereof. The coating, strand, thread or wire is operatively connected to an electrical coupling element (terminal, contact, etc.) on the handle assembly.

Where the instrument is an endoscopic instrument, the tubular member and the shifter member are flexible and dimensioned to fit through a working channel of a flexible endoscope assembly.

Pursuant to another feature of the present invention, the rotation control member is disposed at a distal end of the handle assembly. The rotation control member may take the form of a sleeve or cylinder that is disposed like a nose on the distal end of the handle assembly.

Preferably, the tubular member is fixed at a proximal end to the sleeve, while the shifter member longitudinally traverses the sleeve. The shifter member and the sleeve are rotationally entrained, exemplarily by one or more longitudinal splines fixed to the shifter member and extending into keyways or slots in the sleeve, for instance, in a transverse end wall of the sleeve.

Pursuant to another feature of the present invention, the shifter member is connected to the axial position control member via a wire or rod fixed to the axial position control member, the shifter member being rotatably connected to the wire or rod via a rotary joint disposed inside the sleeve. The rotary joint may be a universal joint such as a ball and socket joint.

The shifter member is preferably connected to the axial position control member via a stiff rod capable of transmitting an axial compressive force without bending or kinking. This rod extends through the handle assembly and the rotary joint (in which case the rod may include two segments coupled to one another via the rotary joint). The rod is generally connected to the shifter member at a point proximate to the rotational control member.

It is to be noted that the operative tip may take virtually any form useful in medical procedures, where it is beneficial to controllably rotate the operative tip to accurately reorient it. For example, the operative may be a cauterization snare or a mechanism with pivoting jaws, such as a grasper, a biopsy forceps, or a stapler. In instruments such as graspers, biopsy forceps or the stapler, it is important that the jaws rotate. The jaws are connected to a clevis, which is connected to an outer sheath of the instrument. So the outer sheath must be rotatable by means a proximal rotator that is part of the handle and located at the distal end of the handle.

### BRIEF DESCRIPTION OF THE DRAWINGS

FIG. 1 is a schematic side elevational view of an endoscopic surgical instrument in accordance with the present invention.

FIG. 2 is a longitudinal cross-sectional view, on a larger scale, of a distal end portion of a handle assembly illustrated in FIG. 1.

FIG. 3 is partially a perspective view and partially a schematic longitudinal cross-sectional view through a distal end of a stapling device rotatable about a longitudinal axis, in accordance with the present invention.

FIG. 4 is a transverse cross-sectional view taken along plane IV-IV in FIG. 3.

FIG. 5 is a schematic perspective view of a handle or actuator that is a part of the stapling device of FIGS. 3 and 4.

### DETAILED DESCRIPTION

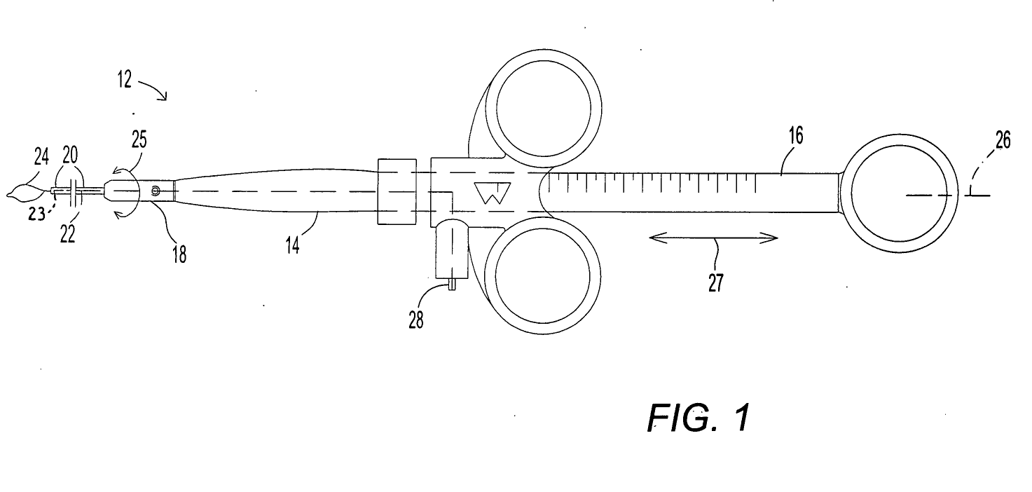

As illustrated in FIG. 1, a medical instrument comprises a handle assembly 12 including a main body 14, an axial position control member 16 translatable relative to the main body, and a rotation control member 18 rotatable relative to the main body 14. An elongate tubular member 20 such as a sheath or catheter is connected at a proximal end to handle assembly 12, and an elongate shifter member 23 in the form of a flexible rod, cable or wire longitudinally traverses the tubular member. An operative tip 24 such as a cauterization loop or snare is connected to shifter member 23 at a distal end thereof. Shifter member 23 is a torque-transmitting rod, wire or cable that is connected at its proximal end, inside sheath 20 near handle assembly 12, to a solid metal rod 22 capable of transmitting compressive forces without kinking.

As further embodiment illustrated in FIG. 1, sheath 20 is operatively connected to rotation control member 18 for rotating about a longitudinal axis 26 in response to manipulation (turning) of the rotation control member. Shifter member or wire 23 is also operatively connected via rod 22 to rotation control member 18 for rotating together with tubular member 20 about longitudinal axis 26 in response to a turning of the rotation control member. Shifter member or wire 23 is operatively connected via rod 22 to axial position control member 16 for shifting axially relative to sheath 20 in response to manipulation of the axial position control member. Thus, the shifter member 23 (e.g., a wire or flexible rod) is rotationally entrained to sheath 20 (e.g., sheath or catheter), but is axially movable relative to sheath 20. Shifter member 23 is rotationally fixed to sheath 20, at the proximal end thereof, for rotation about longitudinal axis 26 in response to a turning of rotation control member 18 as indicated by a double-headed arrow 25. Shifter member 23 is axially or longitudinally movable relative to sheath 20 in response to an axial translation of axial position control member 16 as indicated by an arrow 27.

Preferably, shifter member 23 is made of specially braided stainless steel, torquable in a 1:1 ratio or close to it. If shifter member 23 is made of NiTi, and to the extent that electrical current is to be conducted along the shifter member from handle assembly 12 to operative tip 24, a conductive wire, strand or thread (not separately illustrated) may extend along the nitinol wire 22 for conducting the electrical current. The electrically conductive strand, thread or wire extends along shifter member 23 from a proximal end to a distal end thereof and is connected to operative tip 24 for conveying electrical current thereto. The strand, thread or wire is operatively connected to an electrical coupling element 28 (terminal, contact, etc.) on handle assembly 12.

Where the instrument is an endoscopic instrument, sheath 20 and shifter member 23 are flexible and dimensioned to fit through a working channel of a flexible endoscope assembly.

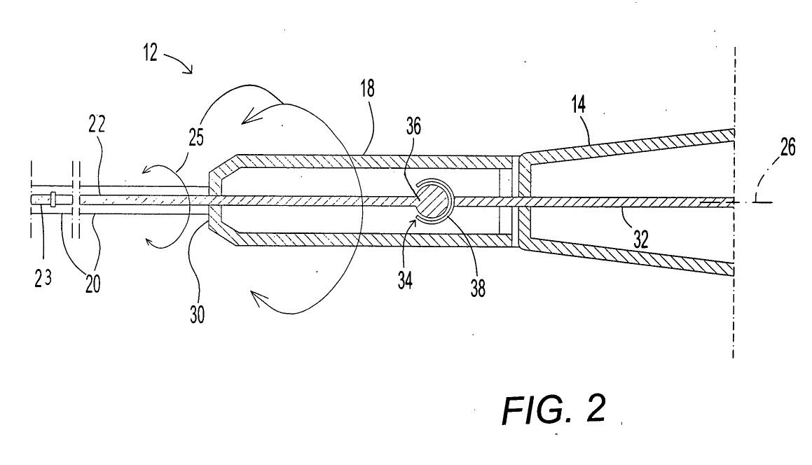

Rotation control member 18 is disposed at a distal end of handle assembly 12. As depicted in FIG. 2, rotation control member 18 specifically takes the form of a sleeve or cylinder that is disposed like a nose on the distal end of handle assembly 12. Sheath 20 is fixed at a proximal end to rotation control sleeve 18, while shifter member 23 longitudinally traverses the sleeve. Shifter member 23 and sleeve 18 are rotationally entrained, exemplarily by one or more longitudinal splines (not shown) fixed to wire 22 and extending into keyways or slots (not shown) in the sleeve, for instance, in a transverse end wall 30 of the sleeve.

As further depicted in FIG. 2, shifter member 23 is connected to axial position control member 18 via rod 22 and another wire or rod 32 fixed to the axial position control member. Rod 22 (and therefore shifter member 23) is rotatably connected to wire or rod 32 via a rotary joint 34 disposed inside the sleeve. Rotary joint 34 particularly takes the form of a universal joint including a ball member 36 and a socket member 38. Rods 22 and 32 may be considered two segments of a rod (not separately designated) that incorporates universal joint 34.

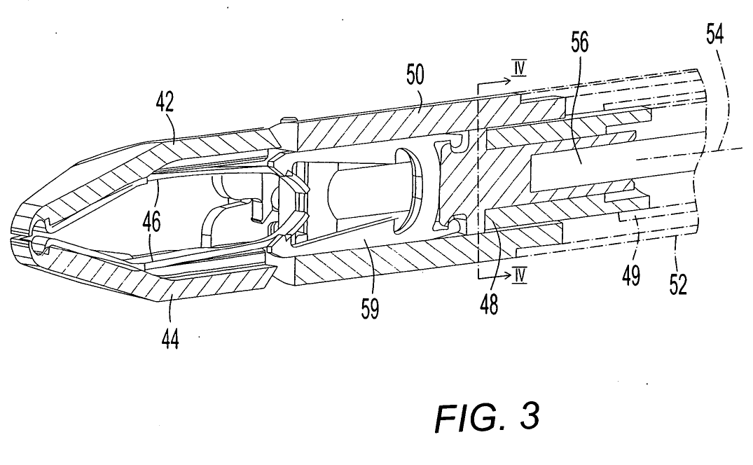

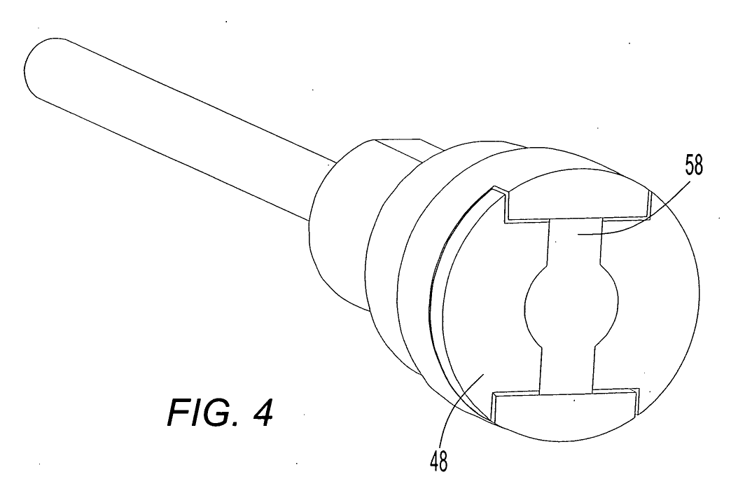

As illustrated in FIGS. 3 and 4, an endoscopic stapling device 40 comprises a pair of stapler jaws 42 and 44 having a loaded pre-firing position in which a staple 46 is disposed in a partially closed configuration between jaws 42 and 44. Jaws 42 and 44 are pivotably secured to a clevis 48 that is in turn fixed to the distal end of a flexible inner tubular sheath or catheter 49. A sleeve 50 for closing jaws 42 and 44 as well as staple 46 is slidably disposed about clevis 48 for advancing over jaws 42 and 44 after the jaws and a staple have been forced over organic tissues at a stapling site inside a patient. Sleeve 50 is fixed at a proximal end to a distal end of a flexible outer tubular sheath or catheter 52. A turning torque for changing the angle of staple jaws 42 and 44 about a longitudinal axis 54 is applied via inner tubular sheath 49 and outer tubular sheath 52. An extractor cable or flexible rod 56 is connected at a distal end to an extractor cap 58 (see FIG. 4). A U-shaped closure bracket or backbone 59 is provided for locking staple 46 in a closed configuration in the patient's tissues. Closure bracket or backbone 59 is advanced over the open staple substantially simultaneously with the closing of jaws 42 and 44.

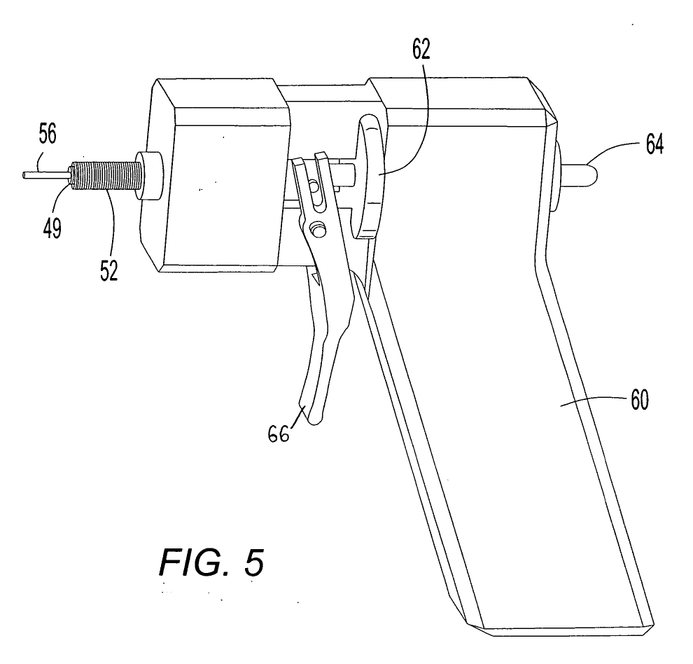

As depicted in FIG. 5, a handle 60 of the stapling device of FIGS. 3 and 4 is connected to the proximal end of extractor cable or rod 56, as well as to inner tubular sheath 49 and outer tubular sheath 52. A thumb wheel 62 is provided on handle 60 for turning inner tubular sheath 49 and outer tubular sheath 52. It is not necessary to turn extractor cable 56 because it is a revolution body without radial referencing. A spring-loaded push button 64 provided at the rear or proximal end of handle 60 is used for ejecting and closing the staple 46.

Outer tubular sheath 52 together with sleeve 50 comprises a shifter member that moves axially or longitudinally in a proximal direction to release jaws 42 and 44 and in a distal direction to close the jaws. Outer tubular sheath 52 is operatively connected inside handle 60 to a lever 66 that is actuated to alternately retract and advance the sheath or catheter 52 to open and close jaws 42 and 44. Sheath 52 is also operatively connected, together with inner tubular sheath 49, to thumb wheel 62 for enabling an operator to control the orientation of an operative tip or end effectuator, i.e., jaws 42 and 44. Extractor cable 56 is operatively connected to push button 64 for pushing closure bracket or backbone 59 about staple 46 to thereby close the staple and lock it in a closed configuration.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A medical instrument comprising:
a handle assembly including a main body, an axial position control member translatable relative to said main body, and a rotation control member rotatable relative to said main body;
an elongate tubular member connected at a proximal end to said handle assembly;
an elongate shifter member coaxial with said tubular member; and
an operative tip connected to one of said tubular member and said shifter member at a distal end thereof,
said tubular member being operatively connected to said rotation control member for rotating about a longitudinal axis in response to manipulation of said rotation control member,
said shifter member also being operatively connected to said rotation control member for rotating about said longitudinal axis in response to manipulation of said rotation control member,
said shifter member being operatively connected to said axial position control member for shifting axially relative to said tubular member in response to manipulation of said axial position control member.

2. The instrument defined in claim 1 wherein said rotation control member is disposed at a distal end of said handle assembly.

3. The instrument defined in claim 2 wherein said rotation control member takes the form of a sleeve.

4. The instrument defined in claim 3 wherein said tubular member is fixed at a proximal end to said sleeve.

5. The instrument defined in claim 4 wherein said shifter member longitudinally traverses said tubular member and is connected to said axial position control member via a wire or rod fixed to said axial position control member, said shifter member being rotatably connected to said wire or rod via a rotary joint.

6. The instrument defined in claim 1 wherein said shifter member longitudinally traverses said tubular member and is connected to said axial position control member via a wire or rod fixed to said axial position control member.

7. The instrument defined in claim 6 wherein said shifter member is rotatably connected to said wire or rod via a rotary joint.

8. The instrument defined in claim 7 wherein said rotary joint is a universal joint.

9. The instrument defined in claim 8 wherein said universal joint is a ball and socket joint.

10. The instrument defined in claim 1 wherein said shifter member longitudinally traverses said tubular member and is made principally of a material taken from the group consisting of a stainless steel and nitinol.

11. The instrument defined in claim 10, wherein said shifter member is made of torquable braided stainless steel.

12. The instrument defined in claim 10, wherein said shifter member is made, at least partially, of another conductive metal.

13. The instrument defined in claim 1 wherein said shifter member longitudinally traverses said tubular member and includes an electrically conductive strand, thread or wire extending along said shifter member from a proximal end to a distal end thereof, said strand, thread or wire being connected to said operative tip for conveying electrical current thereof, said strand, thread or wire being operatively connected to an electrical coupling element on said handle assembly.

14. The instrument defined in claim 1 wherein said tubular member and said shifter member are flexible and dimensioned to fit through a working channel of a flexible endoscope assembly.

15. The instrument defined in claim 1 wherein said operative tip includes a member taken from the group consisting of a flexible loop and stapling jaws.

16. The instrument defined in claim 1 wherein said shifter member longitudinally traverses said tubular member and is connected to said axial position control member via a rod capable of transmitting an axial compressive force without bending or kinking.

17. The instrument defined in claim 1 wherein said tubular member is an inner tubular member, said shifter member being an outer tubular member surrounding said inner tubular member.

18. The instrument defined in claim 17 wherein said operative tip is connected to said inner tubular member.

19. The instrument defined in claim 18 wherein said operative tip includes a pair of jaws spring biased toward an open configuration, said outer tubular member being provided at a distal end with a sleeve engageable with said jaws to close same.

20. A medical instrument comprising:
a handle assembly including a main body, an axial position control member translatable relative to said main body, and a rotation control member rotatable relative to said main body;
an elongate tubular member connected at a proximal end to said handle assembly;
an elongate shifter member longitudinally traversing said tubular member;
an operative tip connected to said shifter member at a distal end thereof; and
an electrical coupling element on said handle assembly,
said shifter member being operatively connected to said rotation control member for rotating about said longitudinal axis in response to manipulation of said rotation control member,
said shifter member being operatively connected to said axial position control member for shifting axially relative to said tubular member in response to manipulation of said axial position control member,
said shifter member being operatively connected to said electrical coupling element and adapted to conduct electrical current from coupling element to said operative tip.

21. The instrument defined in claim 20 wherein said shifter member is connected to said axial position control member via a wire or rod fixed to said axial position control member.

22. The instrument defined in claim 21 wherein said shifter member is rotatably connected to said wire or rod via a rotary joint.

23. A medical instrument comprising:
a handle assembly including a main body, an axial position control member translatable relative to said main body, and a rotation control member rotatable relative to said main body;
an inner tubular member connected at a proximal end to said handle assembly;
an outer tubular member coaxially surrounding said tubular member and connected at a proximal end to said handle assembly; and
an operative tip connected said inner tubular member at a distal end thereof,
said inner tubular member being operatively connected to said rotation control member for rotating about a longitudinal axis in response to manipulation of said rotation control member,
said outer tubular member also being operatively connected to said rotation control member for rotating about said longitudinal axis in response to manipulation of said rotation control member,
said outer tubular member being operatively connected to said axial position control member for shifting axially relative to said tubular member in response to manipulation of said axial position control member.

24. The instrument defined in claim 23 wherein said rotation control member is a thumb wheel.

25. The instrument defined in claim 23 wherein said operative tip is a pair of jaws pivotably connected to said inner tubular member, said jaws being spring biased to assume an opened or spread configuration.

26. The instrument defined in claim 23, further comprising an elongate pusher member longitudinally traversing said inner tubular member, said pusher member being operatively connected at a proximal end to said handle assembly.

27. A medical instrument comprising:
a handle assembly including a main body, an axial position control member translatable relative to said main body, and a rotation control member rotatable relative to said main body;
an elongate tubular member connected at a proximal end to said handle assembly;
an elongate shifter member longitudinally traversing said tubular member; and
an operative tip connected to said shifter member at a distal end thereof,
said tubular member being operatively connected to said rotation control member for rotating about a longitudinal axis in response to manipulation of said rotation control member,
said shifter member also being operatively connected to said rotation control member for rotating about said longitudinal axis in response to manipulation of said rotation control member,
said shifter member being operatively connected to said axial position control member for shifting axially relative to said tubular member in response to manipulation of said axial position control member.
